(19) United States
(12) Patent Application Publication
(10) Pub. No.: US 2026/0325786 A1
(43) Pub. Date: Oct. 22, 2026

(54) DUAL MODALITY THERAPY DEVICES AND METHODS

(71) Applicant: Jeanne RICKS, New Rochelle, NY (US)

(72) Inventor: Jeanne RICKS, New Rochelle, NY (US)

(21) Appl. No.: 19/702,075

(22) Filed: Jun. 9, 2026

Related U.S. Application Data
(63) Continuation of application No. PCT/US2025/010644, filed on Jan. 8, 2025.
(60) Provisional application No. 63/619,048, filed on Jan. 9, 2024.

(51) Int. Cl. A61M 21/00

(52) U.S. Cl. CPC A61M 21/00; A61M 2021/0022; A61M 2021/0027

(57) ABSTRACT

Dual modality therapy devices are disclosed that are operable to provide binaural stimulation and vibroacoustic stimulation along with associated methods. The devices include: a first flexible layer, two or more vibroacoustic transducers, an audio speaker connected to the dual modality therapy device, and a controller electrically coupled to the dual modality therapy device operable to provide at least one vibration control signal to the first vibroacoustic transducer or the second vibroacoustic transducer wherein the first vibroacoustic transducer or the second vibroacoustic transducer vibrates in response to the vibration control signal, and provide at least one binaural beat control signal to the audio speaker for output.

### CROSS-REFERENCE

This application is a continuation of PCT Application PCT/US25/10644 filed Jan. 8, 2025, which claims the benefit of U.S. Provisional Application No. 63/619,048, filed Jan. 9, 2024, ENTITLED VIBROACOUSTIC THERAPY DEVICES AND METHODS

which applications are incorporated herein in their entirety by reference.

### BACKGROUND

### Technical Field

The present disclosure is generally related to dual modality therapy devices and methods operable to deliver vibroacoustic stimulation and binaural stimulation.

Post-traumatic stress disorder (PTSD) affects approximately 3.5% of U.S. adults every year. It is estimated that approximately 6% of the U.S. population will have PTSD at some point in their lives according to the National Center for PTSD at the U.S. Department of Veterans Affairs. Even with the VA estimates there is no single study that asks everyone about PTSD, so the estimates are considered to be inexact. Problems related to PTSD include anger, depression, traumatic brain injury (TBI), chronic pain, sleep problems, and substance abuse or misuse. The estimated cost of treating PTSD was estimated at a staggering $232.2 billion (USD) in 2023. Treatment of PTSD is a significant economic burden which can result in direct health care costs in excess of $26,000 (USD) and 43,000 (Euro) annually. As will be appreciated by those skilled in the art, anger, depression, traumatic brain injury (TBI), chronic pain, sleep problems, and substance abuse or misuse can also occur independent of PTSD and are associated with their own negative economic and societal impacts.

One treatment that has been used for PTSD includes audio therapies. Numerous techniques exist for providing sensory integration for purposes of therapy for the release of stress to improve body functioning. For example, a basic audio system can be used to alter heart rate and biorhythm functioning or to create a soothing effect on an individual.

Vibroacoustic therapies can also be used. Vibroacoustic therapies use audible sound vibrations to reduce symptoms, invoke relaxation, and alleviate stress. Vibroacoustics are based on an understanding that external vibration can influence body function. Vibroacoustic therapy can also be effective for stress, pain management, post-traumatic stress disorder (PTSD), and sleep disorders. Vibroacoustic therapy has also been used for medical conditions, such as cerebral palsy.

Binaural stimulation is a technique that involves a user listening to two different tones played simultaneously in each ear which creates a third tone in the brain. Thus, if a first tone at a first frequency is presented into a first ear and a second tone at a second frequency is presented into a second ear, the brain creates an additional tone that a user can hear. The additional tone is referred to as a binaural beat. Research has found that binaural beats have a positive effect on pain alleviation, anxiety reduction, and memory.

What is needed are dual modality therapy devices operable to deliver coordinated binaural therapy and vibroacoustic therapy and methods that are affordable, portable, and optimized for patient treatment.

### SUMMARY

Disclosed are dual modality therapy devices operable to deliver coordinated binaural therapy and vibroacoustic therapy and associated methods that are portable, affordable and optimized for patient treatment. The disclosed dual modality therapy devices are operable to combine binaural stimulation and vibroacoustic stimulation. Additionally, the dual modality therapy devices can be used with one or more sensors for obtaining additional biometric information, e.g., finger sensor, pulse rate, heart rate etc.

The dual modality therapy devices, systems and methods are configurable to encourage and strengthen a user's natural mechanisms of recovery to, for example, reduce stress, improve sleep, sharpen focus and concentration, lower anxiety, decrease muscular tension and pain, create emotional balance, increase energy level and improve sense of wellbeing.

The dual modality therapy devices, systems and methods operate by using computer-generated programs to induce a frequency following response that is applied to subtle the naturally occurring vibration of the human nervous system. Pure sine waves are used in the frequency range of 30 to 120 Hz. Thousands of clinical research study hours have demonstrated highly statistically significant decreases in blood pressure, pulse rate, anxiety, tension, muscle spasticity, arousal states and dramatic increases in circulation, range of motion, relaxation and sense of well-being and provide other health benefits such as control over pain. Psychological testing shows that people who receive treatments generally feel better.

The treatment serves to remove some of the obstacles that are preventing the body's natural immune system resources from doing their job. Use of the dual modality therapy device also helps reduce symptoms from a wide variety of conditions.

When combined with input from biometric sensors, the information from the biometric sensor can be used to revise the delivery of one or more aspects of the dual modality therapy to the patient.

Both the foregoing general description and the following detailed description are exemplary and explanatory only and are not restrictive of the disclosed embodiments, as claimed.

### INCORPORATION BY REFERENCE

All publications, patents, and patent applications mentioned in this specification are herein incorporated by reference to the same extent as if each individual publication, patent, or patent application was specifically and individually indicated to be incorporated by reference.
FR 2,991,588 B1 issued May 29, 2015 for Method and Device for Emission of Wave and Sensory Stimulations of the Human Body;
IT 202000020959 A1 published Mar. 3, 2022 for Sistema Per Il Trattamento Terapeutico Vibratorio Integrato;
U.S. Pat. No. 9,457,166 issued Oct. 4, 2016 for Physical therapy whole sound frequencies device and method of recording content used therein;
U.S. Pat. No. 9,682,001 issued Jun. 20, 2017 for Wearable bone conduction device;
U.S. Pat. No. 10,912,915 issued Feb. 2, 2021 for Apparatus, system and method of multisensory stimulation;
U.S. Pat. No. 11,541,202 issued Jan. 3 , 2023 for Method, System and Device for Assisted Sleep;
U.S. Pat. No. 11,779,227 issued Oct. 10, 2023 for Multisensory technology for stress reduction and stress resilience;
US 2011/0251535 A1 published Oct. 13, 2011 for Induced Relaxation and Therapeutic Apparatus and Method;
US 2022/0401749 A1 published Dec. 22, 2024 for Device Providing Neurotherapeutic Intervention via Multi-Modal Energy Therapy;

US 2023/0381446 A1 published Nov. 30, 2023 for Method, System and Device for Assisted Sleep;
WO 2013/068569 A2 published May 16, 2013 for Transfer of Structure-Borne Sound Waves Into a Body;
BioMat Health-BioAcoustic Mat (2020);
Mind-Sync Harmonic Sleep Lounger (2023);
Sound Oasis Vibroacoustic Therapy System (2018); and
Welnamis-Binaural Vibroacoustic Technology (2021).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a lateral view of a normal human spinal column; FIG. 1B illustrates the planes of the body identified;

FIG. 2 depicts a human body with locations of vibroacoustic transducers positioned with the therapy device;

FIGS. 3A-3D depicts a therapy device from different views (FIG. 3A top view; FIG. 3B side view along the length of the therapy device; FIG. 3C side view along the top width; and FIG. 3D side view along the bottom width) with vibroacoustic transducers positioned within the therapy device (FIGS. 3A-3C);

FIGS. 4A and 4B depict an alternative configurations of a therapy device with vibroacoustic transducers positioned within the therapy device from a top view;

FIG. 5 depicts another alternative configuration of a therapy device with vibroacoustic transducers positioned within the therapy device from a top view;

FIG. 6 illustrates layers of the therapy device; and

FIG. 7 is a block diagram showing the components of a therapy device.

### DETAILED DESCRIPTION

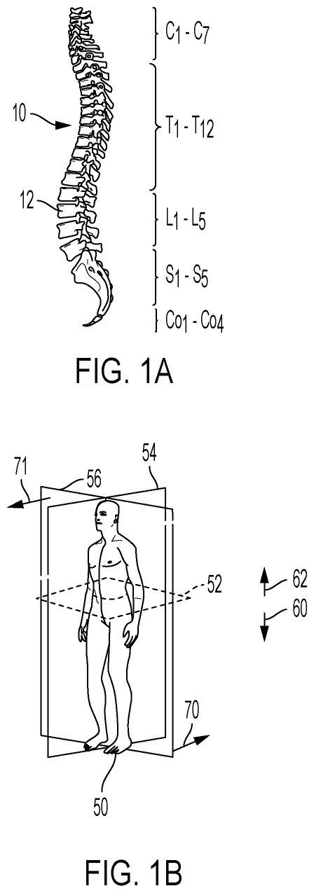

For context, FIG. 1A illustrates the human spinal column 10 which is comprised of a series of thirty-three stacked vertebrae 12 divided into five regions. The cervical region includes seven vertebrae, known as C1-C7. The thoracic region includes twelve vertebrae, known as T1-T12. The lumbar region contains five vertebrae, known as L1-L5. The sacral region is comprised of five fused vertebrae, known as S1-S5, while the coccygeal region contains four fused vertebrae, known as Co1-Co4. The spine has regions that have an inward curvature (lordosis), e.g., the lumbar and cervical regions, and an outward (convex) curvature (kyphosis), e.g., in the thoracic and sacral regions.

In order to understand the configurability, adaptability, and operational aspects of the dual modality therapy devices and methods disclosed herein, it is helpful to understand the anatomical references of the body 50 with respect to which the position and operation of the dual modality therapy devices, and components thereof, are described. FIG. 1B illustrates an overview of a patient 20 of three anatomical planes generally used in anatomy to describe the human body and structure within the human body. The three anatomical planes are: the axial plane 52, the sagittal plane 54 and the coronal plane 56. Additionally, devices and the operation of devices may be better understood with respect to the caudad 60 direction and/or the cephalad direction 62. Devices can be positioned dorsally 70 (or posteriorly) such that the placement or operation of the dual modality therapy device is toward the back or rear of the body. Alternatively, dual modality therapy devices can be positioned ventrally 71 (or anteriorly) such that the placement or operation of the dual modality therapy device is toward the front of the body. Various embodiments of the dual modality therapy devices, systems and tools of the present disclosure may be configurable and variable with respect to a single anatomical plane or with respect to two or more anatomical planes. For example, a subject or a feature of the dual modality therapy device may be described as lying within and having adaptability or operability in relation to a single plane. A dual modality therapy device may be positioned in a desired location relative to a sagittal plane and may be moveable between a number of adaptable positions or within a range of positions.

A variety of users can use the dual modality therapy devices, systems and methods described herein with or without supervision by a medical professional.

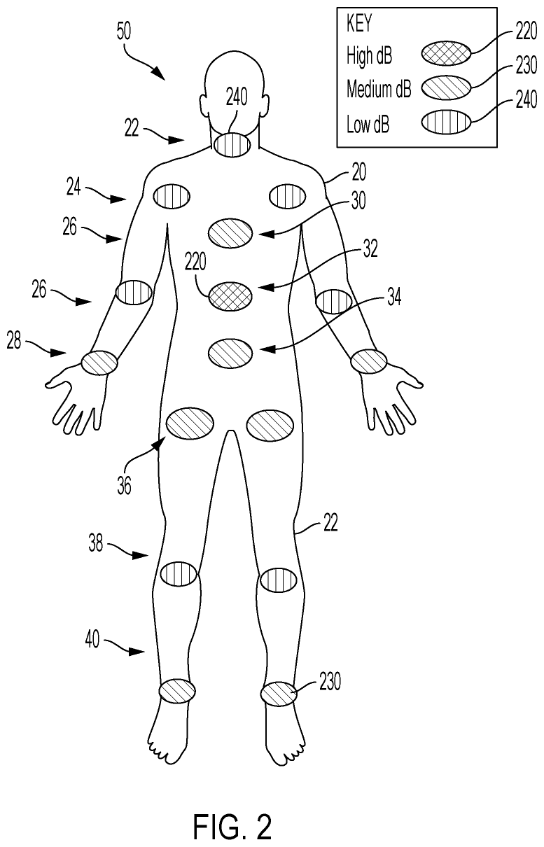

FIG. 2 depicts a human body 50 with associated anatomical locations and intensities of vibroacoustic transducers applied by a dual modality therapy device when the user is laying or sitting on the dual modality therapy device. The vibroacoustic transducers are operable to translate sound a person could hear into sound a person (or user) can feel. The tactile sensation a user experiences is achieved using vibroacoustic transducers. The vibrations permeate through the body to a cellular level. The vibroacoustic transducers can be washable. Additionally, the vibroacoustic transducers can be programmable to create multiple tones at multiple frequencies.

The locations of the vibroacoustic transducers correspond to, for example, the neck 22, the shoulders 24, the elbows 26, the wrists 28, the heart 30, the stomach 32, the bowel 34, the hips 36, the knees 38, and the ankles 40.

The number of vibroacoustic transducers can range from 3 to 25 with two or more db ranges (e.g., high, medium and low; high and medium; high and low; and medium and low). The vibroacoustic transducers can be configured to operate simultaneously (e.g., all vibroacoustic transducers deliver a vibroacoustic signal at the same time), in groups or zones (e.g., vibroacoustic transducers deliver therapy to an anatomical region), in series (e.g., vibroacoustic transducers begin delivering vibroacoustic signal at a first location and then a second location and then a third location), in parallel, or alternately (e.g., vibroacoustic transducers deliver a vibroacoustic signal via a first set of vibroacoustic transducers and then via a second set of vibroacoustic transducers. The series delivery of vibroacoustic therapy can be configured so that only one region or zone is active or one or more prior regions remains active while a new region is activated.

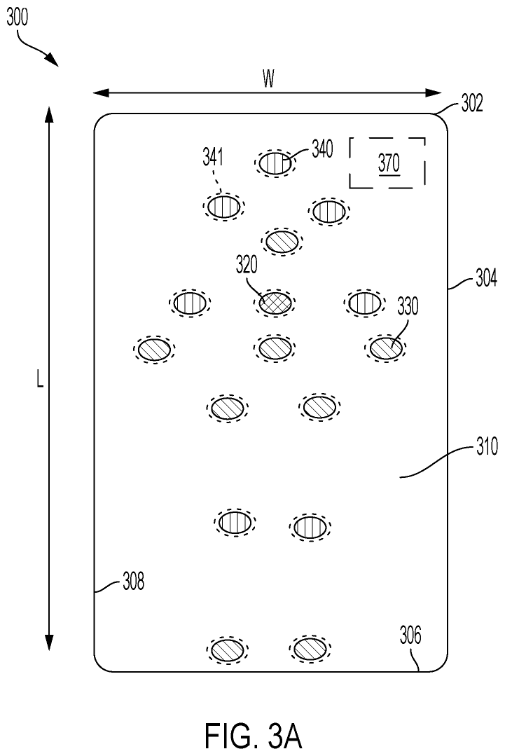

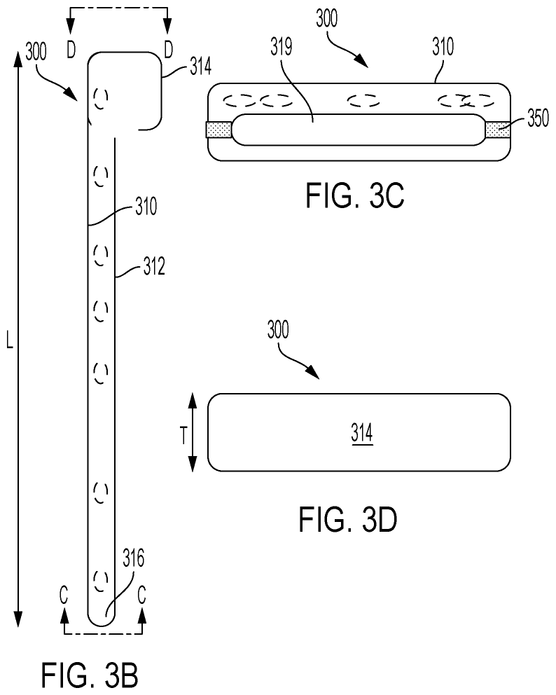

The vibroacoustic transducers are positioned within the dual modality therapy device shown in FIGS. 3-5 so that the position of each vibroacoustic transducer corresponds to a specific anatomical location of a user when the user sits or lays on the dual modality therapy device. As noted, there two or more categories of vibroacoustic transducers: A high db vibroacoustic transducers 220 can have a range of from 55 db to 85 db, more preferably 70 dB. The medium db vibroacoustic transducers 230 can have a range of from 35 db to 65 db, more preferably 50-65 dB. The low db vibroacoustic transducers 240 can have a range of from 30 db to 55 db, more preferably 50 dB.

In some configurations, one high db vibroacoustic transducer 220 is positionable within the dual modality therapy device so that the vibroacoustic transducer is positioned anatomically along the spine 10 at a location between the T1 and T12 vertebra. Two medium db vibroacoustic transducers 230 are positioned within the dual modality therapy device so that a first medium db vibroacoustic transducer 230 is positioned at a location between the T1 and T12 vertebra cephalad to the high db vibroacoustic transducer 220 and a second medium db vibroacoustic transducer 230 is positioned within at a location between the L1 and l% vertebra caudal to the high db vibroacoustic transducer 220.

As shown in FIG. 3A, the high db vibroacoustic transducers 220 can be positioned within the dual modality therapy device to optimize therapy delivery to the stomach 32 area. The medium db vibroacoustic transducers 230 can be positioned within the dual modality therapy device to optimize therapy delivery to the heart 30, the wrist(s) 32, the bowel 34, the hip(s) 36, the knee(s) 38, and the ankle(s) 40. The low db vibroacoustic transducers 240 can be positioned within the dual modality therapy device to optimize therapy delivery to the throat 22, the shoulder(s) 24, the elbow(s) 26, and the knee(s) 38. Additionally, two or more vibroacoustic transducers can be activated as part of an anatomical therapy zone (or zones), e.g., shoulder, hips, right leg, left leg, etc. In this example, the high dB vibroacoustic transducer 220 has an output of about 70 dB, the medium dB vibroacoustic transducer 230 has an output of about 60 dB, and the low dB vibroacoustic transducer 240 has an output of about 50 dB.

One or more additional vibroacoustic transducers are positionable within the dual modality therapy device to provide a low db vibroacoustic transducer 240 positioned anatomically along the spine 10 at a location between the C1 and C7 vertebra cephalad to the first medium vibroacoustic transducer.

One or more vibroacoustic transducers can also be positionable within the dual modality therapy device at locations along the arms 20 of a patient. For example, a first arm location at approximately the shoulder could be the low db vibroacoustic transducer 240, a second arm location at approximately the elbow could be the low db vibroacoustic transducer 240, and a third arm location at approximately the wrist could be a medium db vibroacoustic transducer 230.

One or more vibroacoustic transducers can also be positionable within the dual modality therapy device at locations along the legs 22 of a patient or user. For example, a first leg location at approximately the hip could be a medium db vibroacoustic transducer 230, a second leg location at approximately the knee could be the low db vibroacoustic transducer 240, and a third leg location at approximately the ankle could be a medium db vibroacoustic transducer 230.

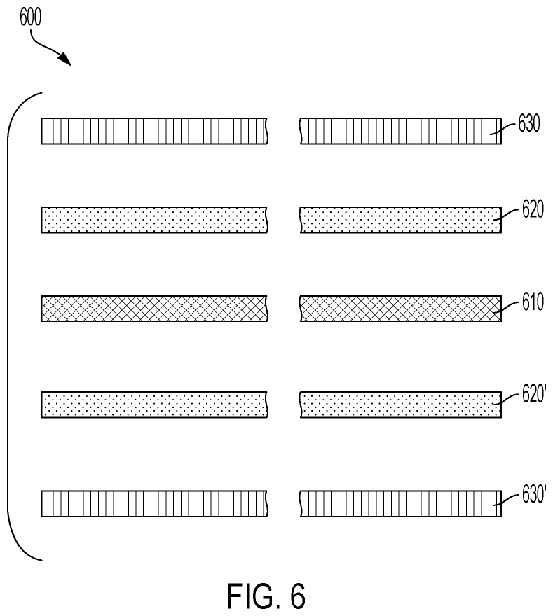

FIGS. 3A-3D depicts a dual modality therapy device 300 from different views (FIG. 3A top view; FIG. 3B side view along the length of the dual modality therapy device; FIG. 3C side view along the top width; and FIG. 3D side view along the bottom width) with vibroacoustic transducers positioned within the dual modality therapy device (FIGS. 3A-3C). The dual modality therapy device 300 is operable to deliver binaural stimulation, i.e., two different tones to each ear simultaneously, while also delivering signals to the body via a vibroacoustic transducers 320, 330, 340. An additional audio track can be delivered with the binaural stimulation. The dual modality therapy device 300 can further include surface indicia corresponding to an optimal location of the vibroacoustic transducers 320, 330, 340 relative to a corresponding anatomy of a patient or user. For example, a body image can be provided on the surface (similar to the body image shown in FIG. 2) or images or markings corresponding to the vibroacoustic transducers 320, 330, 340 themselves can be provided. The dual modality therapy device 300 can be in the form of a mat or pad with a removeable cover that is washable and/or replaceable. The mat or pad can be memory foam, rubber, or cotton. Moreover, the mat or pad can have multiple layers (as shown in FIG. 6) and/or be flexible to allow for rolling or folding into a stored configuration and/or easy transport.

From a top view, the dual modality therapy device 300 has at least one length (L) and at least one width (W) where the length can be greater than the width. For example, the dual modality therapy device 300 can have a rectangular shape in a first plane as illustrated. The length and width can be selected to optimize positioning of the vibroacoustic transducers 320, 330, 340 for users of different heights and widths (e.g., extra small for persons having a height of 5 feet to 5 feet 3 inches, small for persons having a height of from 5 feet 4 inches to 5 feet 7 inches, medium for persons having a height of from 5 feet 8 inches to 6 feet, large for persons having a height of from 6 feet 1 inch to 6 feet 4 inches and extra-large for persons over 6 feet five inches). The dual modality therapy device 300 also has a thickness. The plurality of vibroacoustic transducers 320, 330, 340 are secured to and/or positioned within the dual modality therapy device 300 to deliver vibroacoustic therapy to the desired anatomical location as described herein and with respect to FIG. 2. The dual modality therapy device 300 configuration of FIG. 3 can be used as a mat suitable for placement on the floor, a bed or a chair. FIG. 3B is a side view of the dual modality therapy device 300 along a length (L) and width (W). The dual modality therapy device 300 has an upper surface 310 and a lower surface 312. The upper surface 310 is the surface that engages a user (e.g., the surface upon which a user would lay), the lower surface 312 is the surface that is positioned against a support (e.g., engages the floor, bed or chair). The dual modality therapy device 300 has a top surface 302, a first side surface 304, a bottom surface 306, and a second side surface 308 from a first upper surface planar view (shown in FIG. 3A).

The dual modality therapy device 300 also has a top edge 314 and a bottom edge 316. The top edge 314 can be configured to be thicker than the bottom edge 316 with the thicker end providing, for example, a surface on which a user's head is placed. The plurality of vibroacoustic transducers 320, 330, 340 are shown positioned on or within the dual modality therapy device 300. The vibroacoustic transducers 320, 330, 340 are connected via suitable wiring (not shown). One or more microcontroller 370 is provided that is in communication with the plurality of vibroacoustic transducers 320, 330, 340. The microcontroller 370 can also be in communication with a pair of wired or wireless speakers (not shown). The microcontroller is operable to control the db delivery by the vibroacoustic transducers and/or the wired or wireless speakers. The microcontroller 370 can also include a timer to control the amount of time of a therapy session. Additionally, the microcontroller 370 can include, for example, one or more MP3 players and be removable. The microcontroller 370 is operable to control delivery of waveforms from one or more of the high db tactile transducers 320, medium db tactile transducers 330 and low db tactile transducers 340. The microcontroller 370 is also operable to control delivery of binaural stimulation from a wired or wireless headset.

The view in FIG. 3C is shown from the view of C-C in FIG. 3B. The thicker portion of the dual modality therapy device 300 located at the cephalad most position can be in the form of a pillow positioned within the dual modality therapy device or can be a gap 319 that is created to, for example, allow the cephalad portion of the dual modality therapy device 300 to be positioned over a top rail of a chair. Where the cephalad most portion is operable to fold over the top rail of a chair a securement mechanism 350 can be provided to create an envelope portion that slides over the top rail of the chair. The securement mechanism 350 can be a first flexible surface of loops and a second, opposing flexible surface of hooks (e.g., Velcro®), alternatively, two lengths of flexible material can be provided on either side. The two lengths can either be secured by Velcro or can be tied into a bow or a knot.

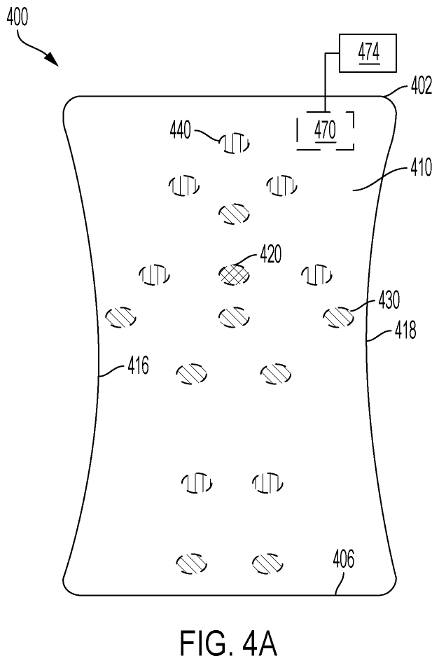

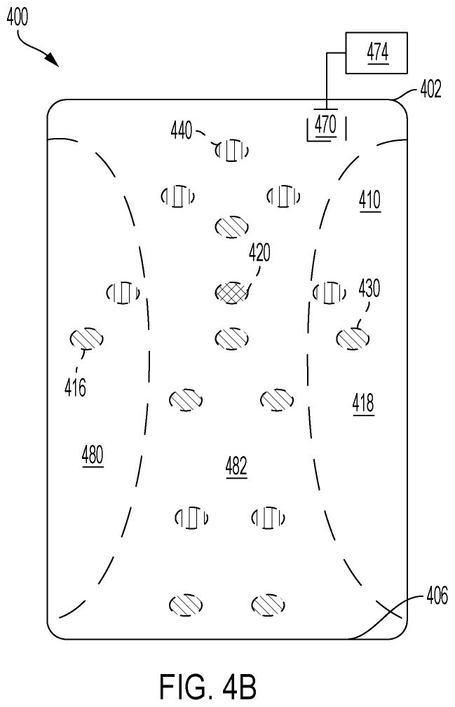

FIG. 4A depicts an alternative configuration of a dual modality therapy device 400 with vibroacoustic transducers 420, 430, 440 positioned within the dual modality therapy device 400 from a top planar view. Microcontroller 470 is shown positioned with the dual modality therapy device 400. The microcontroller 470 can be removably positioned within the dual modality therapy device 400. Additionally, the microcontroller 470 can be wired or wirelessly connected to audible speakers 474 such as a wired or wireless headset. The audible speakers 474 are operable to deliver at least binaural stimulation. The shape of the dual modality therapy device 400 can have an hourglass shape with a first width along the top surface and a second, narrower, width at a location 416, 418 along the length between a top surface 402 and a bottom surface 406. The transition between the first width and the second width can be gradual such that the multiple widths are present along with the length L of the dual modality therapy device 400. The dual modality therapy device 400 of this configuration has a single length and a plurality of widths along the length (e.g., an hourglass shape).

FIG. 4B depicts an alternative configuration of the dual modality therapy device 400 with the vibroacoustic transducers 420, 430, 440 positioned within the dual modality therapy device 400 from a top planar view. Microcontroller 470 is shown positioned with the dual modality therapy device 400. The microcontroller 470 can be removably positioned within the dual modality therapy device 400. Additionally, the microcontroller 470 can be wired or wirelessly connected to audible speakers 474 such as a headset. The thickness of the dual modality therapy device 400 can vary along its length so that an outer portion 480 has a thickness of 0.25 to 0.75 inches, more preferably 0.5 inches and an inner portion 482 has a thickness of 1.5 to 2.5 inches, more preferably 2.0 inches. The shape of an inner portion 482 can be, for example, an hourglass shape with a first width along the top surface and a second, narrower, width at a location 416, 418 along the length between the top surface 402 and the bottom surface 406. The transition between the first width and the second width can be gradual such that the multiple widths are present along with the length L of the dual modality therapy device 400. In another configuration, the change in thickness is achieved by providing layers at some locations and not others (e.g., where the dual modality therapy device is thinner).

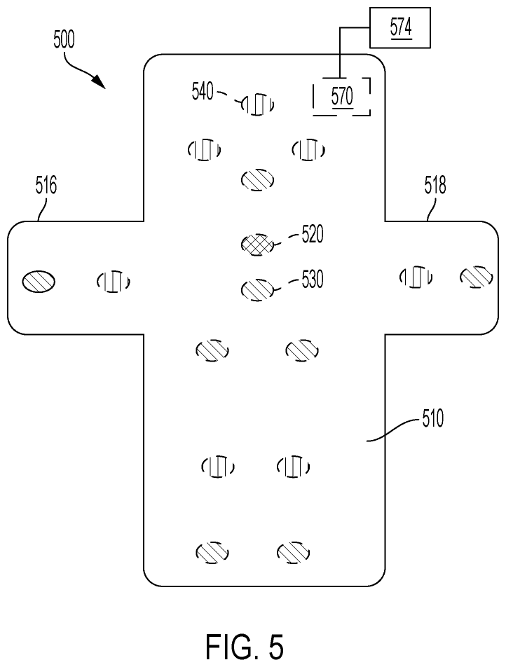

FIG. 5 depicts another alternative configuration of a dual modality therapy device 500 with vibroacoustic transducers 520, 530, 540 positioned within the dual modality therapy device 500 from a top planar view. The configuration has a single length and at least two separate widths. In this configuration, the dual modality therapy device 500 has a T-shape with two arms 516, 518 extending from an elongated section 510. The two arms have one or more vibroacoustic transducers 520, 530, 540 positioned near an edge. This configuration of FIG. 5 would be particularly suitable for use with a chair having arm rests (e.g., a recliner). As will be appreciated by those skilled in the art, the dual modality therapy device 500 can be configured to have a single arm where, for example, the dual modality therapy device 500 is operable to be positioned on a couch or loveseat. As discussed above with respect to FIG. 4, the microcontroller 570 can be removably positioned within the dual modality therapy device 500. Additionally, the microcontroller 570 can be wired or wirelessly connected to audible speakers 574 such as a headset. The audible speakers 574 are operable to deliver at least binaural stimulation.

FIG. 6 illustrates an exploded view layers of the dual modality therapy device 600. A first layer 610 is provided. The first layer can be a layer that provides support for the vibroacoustic transducers. The first layer 610 can have a plurality of apertures or recesses (shown in FIG. 3A, aperture 341) which are operable to affix the vibroacoustic transducers so that the vibroacoustic transducers maintain a position within the dual modality therapy device. Suitable materials for the first layer 610 are, for example, plastic, rubber, thermoplastic elastomer (TPE) and polyvinyl chloride (PVC). One or more second layer 620, 620′ can be provided that is, for example, batting, wadding or cushioning. Suitable batting, wadding or cushioning material includes, natural and synthetic fibers such as cotton, polyester, cotton/poly blend, wool and bamboo. Bonded fibers can be held together with starch or resin/glue. The thickness and choice of material for the second layer is a function of the desired output of the vibroacoustic transducers. One or more third layers 630, 630′ can be provided. The third layer can be a removable and washable bag similar to a duvet cover. In another configuration, the third layer can be an easy to clean surface or disposable exterior surface that operates as a biobarrier. The biobarrier is particularly appropriate where the dual modality therapy device is used in a clinical setting. In some configurations, a single layer can be provided in lieu of the second layer and the third layer that provides both cushioning and operates as a biobarrier.

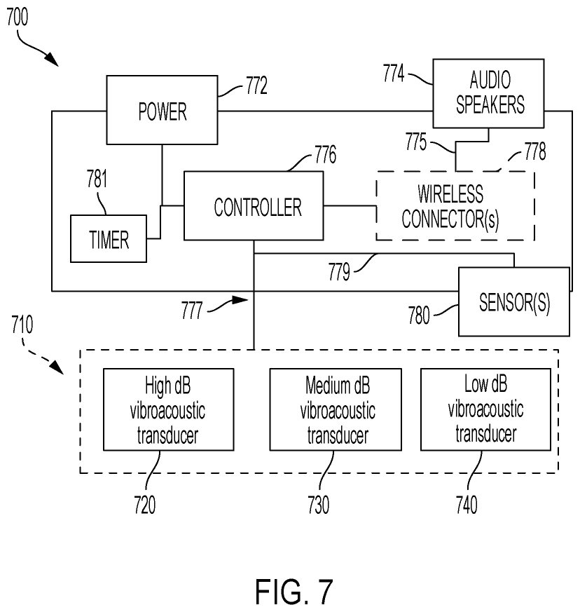

FIG. 7 is a block diagram of the components of a dual modality therapy device 700. The dual modality therapy device 700 has a power supply 772 such as an electrical cord, battery, or rechargeable battery. The power supply 772 is in communication with a controller 776. Audio speakers 774 can be provided which are wired or wireless connected (via wireless connector 778) to the dual modality therapy device 700 and receive input from the controller 776. The wireless connector 778 can also operate to receive software updates and/or transmit data to a remote location. One or more MP3 players can be provided to interface and control the high db vibroacoustic transducer 720, the medium db vibroacoustic transducer 730, the low db vibroacoustic transducer 740, and audio speakers 774.

The controller 776 is operable to control one or more of the vibroacoustic transducers 710 and the audio speakers 774. Audio speakers can be, for example, an external speaker or a pair of audio headphones. An amplifier can also be provided. In some configurations, additional biometric sensors 780 can be provided that are in communication with the controller 776. For example, biometric sensors for sensing the user's pulse rate, heart rate oxygen level, etc. can also be provided without departing from the scope of the disclosure. Information from the one or more biometric sensors can be provided to the controller 776. The controller 776, can then use the biometric sensor information to adjust the dual modality therapy delivered.

The vibroacoustic waveforms delivered by the dual modality therapy devices disclosed herein are broadcast through the body. The waveforms are complex, multilayered audio signal arrays of precise sound frequencies, selected, computer-programmed and balanced for specific therapy applications. The average range of the sound arrays lies between 100 and 1,000 Hertz.

The controller 776 is electrically coupled to the dual modality therapy device operable to provide at least one vibration control signal 777 to the first vibroacoustic transducer or the second vibroacoustic transducer. The signal from the controller 776 causes the receiving vibroacoustic transducer to vibrates in response to the vibration control signal 777. The controller 776 is also operable to provide at least one binaural beat control signal 775 to the audio speaker 774 for output. A timer 781 can also be provided in communication with the controller. The timer 781 can be operable to deliver either or both components of the dual modality therapy for a pre-set amount of time. For example, for sleep, it may be desirable to deliver the dual modality therapy for a specific period of time, e.g., 15 minutes, 30 minutes or 45 minutes.

Soundwaves generated for audio usage are unique brain wave patterns characteristic of specific brain wave states which entrain the brain/mind by repeated use. The audio soundwaves are integrated with the application of the vibroacoustic waves. The combined application of audio and vibroacoustic waveforms are operable to work in conjunction with relaxation techniques for the peak performance training of artists, athletes, meditators, business professionals, lay persons and client populations in stress management, as well as for personal and transpersonal growth. The exercises serve as a powerful and independent training technique for enhancing ‘attentional flexibility’, mental and emotional integration and in the treatment of a number of functional or stress related disorders. A major application of the exercises is for dissolving physical and emotional pain as related to stress.

Suitable components include, but are not limited to: a 15V 3 A Power Supply Adapter Charger Switching Converter Adapter, (Input AC 100V-240V, Output DC 15 Volt 3 Amp 45 Watt), Round Barrel Plug 5.5 mm×2.1 mm & 2.5 mm Tip Center Positive, 6 ft Replacement 3.5 mm Male Plug to Bare Wire Open End TRS 3 Pole Stereo ⅛″ 3.5 mm Plug Jack Connector Audio Cable for Headphone Headset Earphone Cable Repair, USB-A to Micro USB Fast Charging Cable, 480 Mbps Transfer Speed with Gold-Plated Plugs, USB 2.0, Resonance Speaker 50 mm 2 Inch All Frequency Resonance Loudspeaker Vibration Round Strong Bass Vibration Louder speaker (4Ω 25 Watt), DC-DC Automatic Step Up-down Power Module (3~15V to 5V 600 mA), Mini MP3 Player, mini MP3 Player with On-board 128 MB Storage (Breakout), and Dayton Audio DAEX25FHE-4 Framed High Efficiency 25 mm Exciter 24 W 4 Ohm. Suitable transducers include, for example, RoHS 2002/95/EC compliant vibroacoustic transducers available from PUI Audio (https://puiaudio.com/).

### EXAMPLES

During use, when initially activated the low-medium-high outputs of the vibroacoustic transducers are all set at the lowest db level for the category of transducer (e.g., high db tactile transducer at 25 db, medium db tactile transducer at 35 db, and low db tactile transducer at 25 db). The dual modality therapy device is configurable so that the vibroacoustic transducers automatically return to the base (lowest) db level for that vibroacoustic transducer whenever the dual modality therapy device is turned off. A controller is provided that allows the user or practitioner to adjust the db levels from the base (lowest) setting by anatomical region (e.g., left arm, right arm, lower back, upper back, left leg, right leg, spine). Combinations of anatomical regions can also be selected for adjustment without departing from the scope of the disclosure (e.g., right side, right and left arms, etc.). The level of db increase allowed for an anatomical region will be limited to the db levels as already indicated herein. Additionally, even though two anatomical regions may engage a vibroacoustic transducer having the same potential range, the actual applied db range applied can be different. This allows the user to emphasize the vibroacoustic application in body areas of particular concern (e.g., shoulder injury, left knee pain, lower back pain, etc.). The dual modality therapy device is configurable to symptoms. Overexposure can occur if the volume is too strong, from frequency mismatch or an overlong session. Therefore, the dual modality therapy devices and systems are configurable to control the length of a session to avoid overexposure.

Bio Feedback Exercises are designed to teach the user how to manage responses to things like stress and pain via monitor which registers heart rate, typically through a finger sensor.

Controlled Breathing Exercises are designed to calm the nervous system of the user and to control the involuntary functions within the body. Controlled breathing can cause physiological changes that include: lowered heart rate and blood pressure. reduced levels of stress hormones in the blood. Simultaneously, these controlled breathing exercises increase metabolic rate substantially. More oxygen requires more blood flow, although the connection between cerebral metabolism and hemodynamics is not fully understood yet.

Guided Imagery and Visualization Exercises are designed to conduct exercises and giving instruction on visualization of peaceful and calming scenes, which research has shown produce responses in mind and body demonstrated by the reduction in cortisol a stress hormone and increases in hormones shown to improve or enhance mood such as serotonin and endorphins.

Researchers report that visualization and controlled breathing exercises induce positive structural changes in brain connectivity by boosting efficiency in a part of the brain that helps a person regulate behavior in accordance with their goals. A type of magnetic resonance called diffusion tensor imaging allowed researchers to examine fibers connecting brain regions before and after this type of training. The changes were strongest in connections involving the anterior cingulate.

### A. Relaxation

A two-tiered relaxation protocol can include application of tactile vibroacoustic transducers delivered through a mat, furniture and/or integrated wearables, broadcasting biometrical sound frequencies to be focused using the microcontroller on the infraspinatus muscle, which covers the outside of the shoulder blades. A starting point low amplitude in the target area for relaxation will be raised from 25 db to 44 db at specific frequency ranges of between 52 hz and 68 hz to provide both spasmolytic and muscle relaxing effect, increased blood circulation, and positively affect the autonomic and para-sympathetic nervous systems. Simultaneously, binaural stimulation can be used aurally to reduce stress levels and help to break the stress and pain cycle which will enhance pain reduction.

By using binaural stimulation to invoke the frequency following response this therapeutic protocol helps the brain downshift from a beta state (normal waking consciousness) to the alpha state (relaxed consciousness), and even the theta (meditative state) and delta (sleep, when internal healing occurs. Binaural stimulation promote positive neuroplasticity, by employing soundwaves, frequencies modulation, and music to enable the brain to better manage and reduce the production of, and the negative effects of continual stimulation of the adrenal cortex causing over-production of cortisol.

The two-tiered protocol includes application of tactile vibroacoustic frequencies delivered through a mat, furniture and/or integrated wearables, broadcasting biometrical sound frequencies to be focused using the microcontroller to deliver tones through the body at varying rates with a combination of 6 frequencies in ranges of 30 hz and 68 hz. Simultaneously, binaural stimulation can be used aurally starting at 35 hz and cycling down to 8 hz or 4 hz.

### B. Sleep (Inducement or Improvement)

A timed two-tiered relaxation protocol can include application of tactile vibroacoustic transducers delivered through a mat, furniture and/or integrated wearables, broadcasting biometrical sound frequencies to be focused using the microcontroller on the infraspinatus muscle, which covers the outside of the shoulder blades and also down the Latissimus dorsi. A starting point low amplitude in the target area for relaxation will be raised from 25 db to 44 db at specific frequency ranges of between 52 hz and 68 hz to provide both spasmolytic and muscle relaxing effect, increased blood circulation, and positively affect the autonomic and para-sympathetic nervous systems. Simultaneously, binaural stimulation can be used aurally to reduce stress levels and help to break the stress and pain cycle which will enhance pain reduction. The timed application can be, for example, 15 minutes, 30 minutes, or 45 minutes.

As noted above, by using binaural stimulation to invoke the frequency following response this therapeutic protocol helps the brain downshift from a beta state (normal waking consciousness) to the alpha state (relaxed consciousness), and even the theta (meditative state) and delta (sleep, when internal healing occurs).

### C. Shoulder Injury

A two-tiered protocol for a shoulder injury includes application of tactile vibroacoustic frequencies delivered through a mat, furniture and/or integrated wearables, broadcasting biometrical sound frequencies to be focused using the microcontroller on the infraspinatus muscle, which covers the outside of the shoulder blades. A starting point low amplitude in that area is raised from 25 db to 44 db at specific frequency ranges of between 52 hz and 68 hz to provide both spasmolytic and muscle relaxing effect, increased blood circulation, and positively affect the autonomic and para-sympathetic nervous systems. Simultaneously, binaural waves can be used aurally to reduce stress levels and help to break the stress and pain cycle which will enhance pain reduction.

### D. Pain Management

Relaxing the musculature and increasing blood flow are underlying mechanisms for reaching the goals of pain relief. Many dealing with chronic pain struggle with colloquially named ‘Painsomnia’ (insomnia caused by pain). Yet, sleep is essential for healing. Lack of sleep not only exacerbates pain symptoms leading to chronic pain~it is also detrimental to general physical and mental health. Invoking the relaxation response via application of vibroacoustic frequencies is another key for pain mitigation.

Research has shown that vibroacoustic therapies can work within the effective range of a vibration-induced, natural pain-suppressing mechanism of the (Pacinian Corpuscles) pressure-sensitive nerve endings located in the subcutaneous and connective tissues surrounding visceral organs and joints.

Pain causes a sustained endocrine stress response, which in turn induces changes in the function of the hippocampal complex that may contribute to a persistent pain state. Fear of being in pain, the avoidance and anticipation of pain, can lead to a behavior change known as catastrophizing. Chronic pain changes how your brain and nervous system work, which is known as central sensitization, wherein your nervous system is in a constant state of overdrive. This is an ongoing cycle where pain causes stress and stress causes pain, and so it causes its own self-repeating loop. A therapeutic approach combining vibroacoustic therapy with binaural stimulation is effective to induce muscle relaxation, increase blood flow and reduce underlying mental stress mechanisms.

A two-tiered pain management protocol includes application of tactile vibroacoustic frequencies delivered through a mat, furniture and/or integrated wearables, broadcasting biometrical sound frequencies to be focused using the microcontroller to deliver tones through the body evenly at 45 db at using a combination of 6 frequencies in ranges of 30 hz and 68 hz to provide both spasmolytic and muscle relaxing effect, increased blood circulation, and positively affect the autonomic and para-sympathetic nervous systems. Concurrently, binaural stimulation can be broadcast aurally to reduce stress levels and help to break the stress and pain cycle which will enhance pain reduction.

A memory can be provided, which may be internal memory or external memory to microcontroller, may be implemented in firmware and/or software implementation. The firmware and/or software implementation methodologies may be implemented with modules (e.g., procedures, functions, and so on) that perform the functions described herein. A machine-readable medium tangibly embodying instructions may be used in implementing the methodologies described herein. For example, software codes may be stored in a memory and executed by a processor unit (e.g., microcontroller). Memory may be implemented within the processor unit or external to the processor unit. As used herein, the term “memory” refers to types of long term, short term, volatile, nonvolatile, and/or other non-transitory memory and is not to be limited to a particular type of memory or number of memories, or type of media upon which memory is stored.

If implemented in firmware and/or software, and/or as part of microcontroller and/or memory, the functions described herein may be stored as one or more instructions or code on a computer-readable medium. Examples include computer-readable media encoded with a data structure and computer-readable media encoded with a computer program. Computer-readable media includes physical computer storage media. A storage medium may be an available medium that can be accessed by a computer. By way of example, and not limitation, such computer-readable media can include RAM, ROM, EEPROM, CD-ROM or other optical disk storage, magnetic disk storage or other magnetic storage devices, or other medium that can be used to store desired program code in the form of instructions or data structures and that can be accessed by a computer (e.g., microcontroller); disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

In addition to storage on computer readable medium, instructions and/or data may be provided as signals on transmission media included in a communication apparatus. For example, a communication apparatus may include a transceiver having signals indicative of instructions and data. The instructions and data are configured to cause one or more processors (e.g., microcontroller) to implement the functions outlined.

Those of skill would further appreciate that the various illustrative logical blocks, modules, circuits, and algorithm steps described in connection with the disclosure herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The various illustrative logical blocks, modules, and circuits described in connection with the disclosure herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, multiple microprocessors, one or more microprocessors in conjunction with a DSP core, or any other such configuration.

In one or more exemplary designs, the functions described may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. A storage media may be any available media that can be accessed by a general purpose or special purpose computer. By way of example, and not limitation, such computer-readable media can include RAM, ROM, EEPROM, CD-ROM or other optical disk storage, magnetic disk storage or other magnetic storage devices, or any other medium that can be used to carry or store specified program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

The present teachings may also extend to one or more of the following numbered clauses:

Clause 1. A dual modality therapy device 300, 400, 500, 600,700 for providing binaural stimulation and vibroacoustic stimulation comprising:
a first flexible layer 310, 410, 510, 610 having a length L and a width W;
two or more vibroacoustic transducers 320, 330, 340 comprising
a first vibroacoustic transducer 320, 330, 340 affixed to the first flexible layer 310, 410, 510 at a first location, wherein the first vibroacoustic transducer is selected from a high db vibroacoustic transducer 220, a medium db vibroacoustic transducer 230, and a low db vibroacoustic transducer 240;
a second vibroacoustic transducer affixed to the first flexible layer 310, 410, 510, 610 at a second location, wherein the second vibroacoustic transducer is selected from a high db vibroacoustic transducer 220, a medium db vibroacoustic transducer 230, and a low db vibroacoustic transducer 240, wherein the first vibroacoustic transducer 320, 330, 340 and the second vibroacoustic transducer 320, 330, 340 are selected from different groups;
an audio speaker 774 connected to the dual modality therapy device 300, 400, 500, 600, 700; and
a controller 776 electrically coupled to the dual modality therapy device operable to
provide at least one vibration control signal 777 to the first vibroacoustic transducer or the second vibroacoustic transducer wherein the first vibroacoustic transducer or the second vibroacoustic transducer vibrates in response to the vibration control signal 777, and provide at least one binaural beat control signal 775 to the audio speaker 774 for output.

Clause 2. The dual modality therapy device 300, 400, 500, 600,700 of clause 1, wherein the vibration control signal 777 is responsive to a biofeedback sensor 780 or a neurofeedback sensor 780.

Clause 3. The dual modality therapy device 300, 400, 500, 600,700 of clause 1, further comprising a third transducer affixed to the first flexible layer 310, 410, 510, 610 at a third location, wherein the third transducer is selected from the high db vibroacoustic transducer 220, the medium db vibroacoustic transducer 230, and the low db vibroacoustic transducer 240.

Clause 4. The dual modality therapy device 300, 400, 500, 600,700 of clause 1, further comprising three or more additional vibroacoustic transducers 320, 330, 340.

Clause 5. The dual modality therapy device 300, 400, 500, 600,700 of clause 1, wherein one or more vibroacoustic transducers 320, 330, 340 are affixed to the first flexible layer 310, 410, 510, 610 at locations corresponding to a patient anatomy.

Clause 6. The dual modality therapy device 300, 400, 500, 600,700 of clause 5, wherein the one or more vibroacoustic transducers 320, 330, 340 are affixed to the first flexible layer 310, 410, 510, 610 at a dual modality therapy device location corresponding to one or more of an ankle 40, a knee 38, a hip 36, a bowel 34, a stomach 32, a heart 30, a wrist 28, an elbow 26, a shoulder 24 and a neck 22.

Clause 7. The dual modality therapy device 300, 400, 500, 600,700 of clause 1 wherein the at least one vibration control signal 777 and the at least one binaural beat control signal 775 is delivered simultaneously by the controller 776 to the vibroacoustic transducers 320, 330, 340 and the audio speaker 774.

Clause 8. The dual modality therapy device 300, 400, 500, 600,700 of clause 1 wherein the at least one vibration control signal 777 and the at least one binaural beat control signal 775 is delivered serially by the controller 776 to the vibroacoustic transducers 320, 330, 340 and the audio speaker 774.

Clause 9. The dual modality therapy device 300, 400, 500, 600,700 of clause 1 wherein the at least two vibration control signals 777 are delivered by the controller 776 in an alternating pattern to the two or more vibroacoustic transducers 320, 330, 340.

Clause 10. The dual modality therapy device 300, 400, 500, 600,700 of clause 1 wherein the controller 776 receives a biometric input from one or more biometric sensors 780.

Clause 11. The dual modality therapy device 300, 400, 500, 600,700 of clause 10 wherein one or more of a vibration control signal 777 and a binaural beat control signal 775 is altered based on the biometric input received from the one or more biometric sensors 780.

Clause 12. A therapy method comprising:
providing a dual modality therapy device 300, 400, 500, 600,700 comprising a first flexible layer 310, 410, 510, 610 having a length L and a width W, two or more vibroacoustic transducers 320, 330, 340 comprising a first vibroacoustic transducer 320, 330, 340 affixed to the first flexible layer 310, 410, 510, 610 at a first location, wherein the first vibroacoustic transducer 320, 330, 340 is selected from a high db vibroacoustic transducer 220, a medium db vibroacoustic transducer 230, and a low db vibroacoustic transducer 240, a second vibroacoustic transducer 320, 330, 340 affixed to the first flexible layer 310, 410, 510, 610 at a second location, wherein the second vibroacoustic transducer 320, 330, 340 is selected from a high db vibroacoustic transducer 220, a medium db vibroacoustic transducer 330, and a low db vibroacoustic transducer 340, wherein the first vibroacoustic transducer 320, 330, 340 and the second vibroacoustic transducer 320, 330, 340 are selected from different groups, and a controller electrically coupled to the dual modality therapy device;
delivering at least one vibration control signal 777 from the controller 776 to the first vibroacoustic transducer 320, 330, 340 or the second vibroacoustic transducer 320, 330, 340; and
vibrating the first vibroacoustic transducer 320, 330, 340 or the second vibroacoustic transducer 320, 330, 340 in response to the vibration control signal 777 from the controller 776.

Clause 13. The therapy method of clause 12 further comprising: connecting a pair of headphones to the dual modality therapy device 300, 400, 500, 600,700; and
delivering at least one binaural beat control signal 775 from the controller to the audio speakers 774.

Clause 14. The therapy method of clause 13 wherein the at least one vibration control signal 777 and the at least one binaural beat control signal 775 is delivered simultaneously by the controller 776 to the two or more vibroacoustic transducers 320, 330, 340 and the audio speaker 774.

Clause 15. The therapy method of clause 13 wherein the at least one vibration control signal 777 and the at least one binaural beat control signal 775 is delivered serially by the controller 776 to the two or more vibroacoustic transducers 320, 330, 340 and the audio speaker 774.

Clause 16. The therapy method of clause 12 wherein vibroacoustic transducers 320, 330, 340 are affixed to the first flexible layer 310, 410, 510, 610 at locations corresponding to one or more of an ankle, a knee, a hip, a bowel, a stomach, a heart, a wrist, an elbow, a shoulder and a neck.

Clause 17. The therapy method of clause 16 wherein the controller delivers a control signal to the transducers to deliver vibroacoustic signals to one or more of the ankle 40, the knee 38, the hip 36, the bowel 34, the stomach 32, the heart 30, the wrist 28, the elbow 26, the shoulder 24 and the neck 22.

Clause 18. The therapy method of clause 12 wherein the at least two vibration control signals 777 are delivered by the controller 776 in an alternating pattern to the two or more vibroacoustic transducers 320, 330, 340.

Clause 19. The therapy method of clause 12 further comprising identifying an anatomical therapy zone and delivering vibration control signals 777 to one or more vibroacoustic transducers 320, 330, 340 located within the anatomical therapy zone.

Clause 20. The therapy method of clause 12 further comprising receiving a biometric input from the one or more biometric sensors 780 and adjusting one or more of a vibration control signal 777 and a binaural beat control signal 775 based on the biometric input 779 from the one or more biometric sensors 780.

While preferred embodiments of the present invention have been shown and described herein, it will be obvious to those skilled in the art that such embodiments are provided by way of example only. Numerous variations, changes, and substitutions will now occur to those skilled in the art without departing from the invention. For example, the use of comprise, or variants such as comprises or comprising, includes a stated integer or group of integers but not the exclusion of any other integer or group of integers. It should be understood that various alternatives to the embodiments of the invention described herein may be employed in practicing the invention. It is intended that any claims presented define the scope of the invention and that methods and structures within the scope of these claims and their equivalents be covered thereby.

What is claimed:

1. A dual modality therapy device for providing binaural stimulation and vibroacoustic stimulation comprising:
a first flexible layer having a length and a width;
two or more vibroacoustic transducers comprising
a first vibroacoustic transducer affixed to the first flexible layer at a first location, wherein the first vibroacoustic transducer is selected from a high db vibroacoustic transducer, a medium db vibroacoustic transducer, and a low db vibroacoustic transducer;
a second vibroacoustic transducer affixed to the first flexible layer at a second location, wherein the second vibroacoustic transducer is selected from a high db vibroacoustic transducer, a medium db vibroacoustic transducer, and a low db vibroacoustic transducer, wherein the first vibroacoustic transducer and the second vibroacoustic transducer are selected from different groups;
an audio speaker connected to the dual modality therapy device; and
a controller electrically coupled to the dual modality therapy device operable to
provide at least one vibration control signal to the first vibroacoustic transducer or the second vibroacoustic transducer wherein the first vibroacoustic transducer or the second vibroacoustic transducer vibrates in response to the vibration control signal, and
provide at least one binaural beat control signal to the audio speaker.

2. The dual modality therapy device of claim 1, wherein the vibration control signal is responsive to a biofeedback sensor or a neurofeedback sensor.

3. The dual modality therapy device of claim 1, further comprising a third transducer affixed to the first flexible layer at a third location, wherein the third transducer is selected from the high db vibroacoustic transducer, the medium db vibroacoustic transducer, and the low db vibroacoustic transducer.

4. The dual modality therapy device of claim 1, further comprising three or more vibroacoustic transducers.

5. The dual modality therapy device of claim 1, wherein one or more vibroacoustic transducers are affixed to the first flexible layer at locations corresponding to a patient anatomy.

6. The dual modality therapy device of claim 5, wherein the one or more vibroacoustic transducers are affixed to the first flexible layer at dual modality therapy device location corresponding to one or more of an ankle, a knee, a hip, a bowel, a stomach, a heart, a wrist, an elbow, a shoulder and a neck.

7. The dual modality therapy device of claim 1 wherein the at least one vibration control signal and the at least one binaural beat control signal is delivered simultaneously by the controller to the vibroacoustic transducers and the audio speaker.

8. The dual modality therapy device of claim 1 wherein the at least one vibration control signal and the at least one binaural beat control signal is delivered serially by the controller to the vibroacoustic transducers and the audio speaker.

9. The dual modality therapy device of claim 1 wherein the at least two vibration control signals are delivered by the controller in an alternating pattern to the vibroacoustic transducers.

10. The dual modality therapy device of claim 1 wherein the controller receives an input from one or more sensors.

11. The dual modality therapy device of claim 10 wherein one or more of a vibration control signal and a binaural beat control signal is altered based on the input received from the one or more sensors.

12. A therapy method comprising:
providing a dual modality therapy device comprising a first flexible layer having a length and a width, two or more vibroacoustic transducers comprising a first vibroacoustic transducer affixed to the first flexible layer at a first location, wherein the first vibroacoustic transducer is selected from a high db vibroacoustic transducer, a medium db vibroacoustic transducer, and a low db vibroacoustic transducer, a second vibroacoustic transducer affixed to the first flexible layer at a second location, wherein the second vibroacoustic transducer is selected from a high db vibroacoustic transducer, a medium db vibroacoustic transducer, and a low db vibroacoustic transducer, wherein the first vibroacoustic transducer and the second vibroacoustic transducer are selected from different groups, and a controller electrically coupled to the dual modality therapy device;
delivering at least one vibration control signal from the controller to the first vibroacoustic transducer or the second vibroacoustic transducer; and
vibrating the first vibroacoustic transducer or the second vibroacoustic transducer in response to the vibration control signal from the controller.

13. The therapy method of claim 12 further comprising:
connecting a pair of headphones to the dual modality therapy device; and
delivering at least one binaural beat control signal from the controller to the pair of headphones.

14. The therapy method of claim 13 wherein the at least one vibration control signal and the at least one binaural beat control signal is delivered simultaneously by the controller to the two or more vibroacoustic transducers and the audio speaker.

15. The therapy method of claim 13 wherein the at least one vibration control signal and the at least one binaural beat control signal is delivered serially by the controller to the two or more vibroacoustic transducers and the audio speaker.

16. The therapy method of claim 12 wherein transducers are affixed to the first flexible layer at locations corresponding to one or more of an ankle, a knee, a hip, a bowel, a stomach, a heart, a wrist, an elbow, a shoulder and a neck.

17. The therapy method of claim 16 wherein the controller delivers a control signal to the transducers to deliver vibroacoustic signals to one or more of the ankle, the knee, the hip, the bowel, the stomach, the heart, the wrist, the elbow, the shoulder and the neck.

18. The therapy method of claim 12 wherein the at least two vibration control signals are delivered by the controller in an alternating pattern to the two or more vibroacoustic transducers.

19. The therapy method of claim 12 further comprising identifying an anatomical therapy zone and delivering vibration control signals to one or more vibroacoustic transducers located within the anatomical therapy zone.

20. The therapy method of claim 12 further comprising receiving an input from one or more sensors and adjusting one or more of a vibration control signal and a binaural beat control signal based on the input from the one or more sensors.
